(19) United States
(12) Patent Application Publication
(10) Pub. No.: US 2025/0059573 A1
(43) Pub. Date: Feb. 20, 2025

(54) CARBON FIXATION SYSTEM

(71) Applicant: Phase Biolabs Ltd., Nottingham Nottinghamshire (GB)

(72) Inventor: David Ortega, Nottingham Nottinghamshire (GB)

(21) Appl. No.: 18/720,403

(22) PCT Filed: Dec. 20, 2022
(86) PCT No.: PCT/EP2022/086989

§ 371 Date: Jun. 14, 2024

(30) Foreign Application Priority Data
Dec. 23, 2021 (EP) 21217564.0
Dec. 24, 2021 (EP) 21217703.4

(51) Int. Cl. C12P 13/02; C12N 9/02; C12R 1/02

(52) U.S. Cl. CPC C12P 13/02; C12N 9/0004; C12N 2510/02; C12R 2001/02

(57) ABSTRACT

The present invention relates to carbon fixation systems, wherein an inorganic carbon source is converted to acetyl-CoA. A system for the generation of acetyl-Coenzyme A (acetyl-CoA), comprising components necessary for the biochemical conversion of an inorganic carbon source into acetyl-CoA, wherein said components comprise:
i. a source of reducing equivalents;
ii. a light-dependent ion pump that generates an electrochemical ion gradient independently of the generation of reducing equivalents; and
iii. a redox-dependent ion pump that generates an electrochemical ion gradient independently of a net change in the number of reducing equivalents.

### FIELD OF THE INVENTION

The present invention relates to a system for carbon fixation. In particular, the present invention relates to the components necessary for the conversion of an inorganic carbon source, such as CO₂, into multi-carbon compounds via acetyl-CoA.

### BACKGROUND OF THE INVENTION

There is a strong link between increasing global temperatures and the rising concentration of greenhouse gases in the atmosphere. Carbon dioxide (CO₂) is a major constituent of greenhouse gases. Currently, the energy sector is the largest contributor globally to CO₂ emissions, particularly in the production of electricity and heat [1].

Carbon fixation is the process of converting inorganic carbon, such as CO₂, into organic compounds, and can occur by both non-biological and biological mechanisms. In all biological cases, carbon fixation requires two cellular resources: energy in the form of adenosine triphosphate (ATP), and a source of electrons in the form of reducing equivalents. Carbon atoms in CO₂ molecules exist in their highest oxidation state, while those in common fuels and chemicals such as hydrocarbons, alcohols, and acids are in lower states. Energy input is thus required for the reduction of this carbon in order to synthesize organic carbon from inorganic carbon sources. ATP is the universal energy carrier for all known life forms, wherein hydrolysis of a high-energy phosphate bond is carried out to release stored energy. Reducing equivalents are chemical species involved in reduction-oxidation (redox) reactions that donate electrons in order to reduce the electron accepting species. The mechanisms for generation of ATP can be linked to the generation or consumption of reducing equivalents, or not.

Non-biological carbon fixation can be achieved through a thermochemical process called Fischer-Tropsch, which converts a mixture of carbon monoxide (CO) and hydrogen gas (H₂) (known as syngas) into liquid hydrocarbons. Syngas can be derived from various sources, including solid substrate gasification and gaseous substrate gasification. Although Fischer-Tropsch carbon fixation is a commercial process, it necessitates operation at high temperature and pressure, resulting in large expenses and poor efficiencies.

Non-biological carbon fixation may also proceed via electrochemical catalysis, whereby energy is supplied via electricity and materials, including metal alloys, noble metals and carbon nano-sheets, which act as the catalyst for CO₂ fixation. Electrochemical catalysts have not yet been commercialised due to large overpotentials, slow electron transfer kinetics, poor selectivity and rapid degradation of the catalyst.

Finally, non-biological carbon fixation may also proceed via photochemical catalysis whereby semiconducting materials, metals, or metal oxides can act as the catalyst, in addition to combined metal catalysts, metal-free catalysts and Z-scheme catalysts. These catalysts absorb energy from radiated light to drive CO₂ fixation. Carbon fixation using photochemical catalysis has not been commercialised due to poor yields, poor light absorption and poor stability of the catalyst caused by high charge recombination, inappropriate or wide band gaps, slow electron transfer and photo-corrosion.

Biological carbon fixation is carried out by autotrophic organisms that employ native carbon fixation pathways to assimilate taken up CO₂ into the cellular metabolites required for biomass formation and growth. There are two types of autotropic organisms: phototrophs and chemotrophs. Phototrophs use energy from captured light to oxidise an inorganic substrate, which is where they derive their electrons from. In contrast, chemotrophs derive both their energy and electrons from inorganic sources, such as H₂.

Six carbon fixation pathways have been discovered in nature, four of which are cyclical and two of which are linear. Namely, the Calvin-Benson-Bassham (CBB) cycle, the 3-hydroxypropionate (3HP) bicycle, the 3-hydroxypropionate-4-hydroxybutyrate (3HP/4HB) cycle, the reductive tricarboxylic acid (rTCA) cycle, the dicarboxylate/4-hydroxybutyrate (4HP) cycle and the Wood-Ljungdahl pathway (WLP). Phototrophs such as plants and green non-sulphur bacteria use the CBB cycle or the 3HP bicycle, whereby energy derived from light is used to extract electrons from water. In organisms that use the rTCA for carbon fixation, such as prokaryotes classified in the Thermoproteus genus, energy derived from light is used to generate reducing equivalents by extracting electrons from inorganic sources like H₂S, Fe²⁺. Microorganisms using the 3HP/4HB cycle and the 4HP cycle can extract energy and electrons from inorganic sources such as H₂, sulphur (S) or metal ions (Fe²⁺). The WLP is found only in anaerobic bacteria such as acetogens. Acetogenic microorganisms couple the oxidation of inorganic substrates such as H₂ or CO to the reduction of CO₂ or CO into acetyl-CoA, which is converted into acetate.

Reported synthetic biological carbon fixation pathways include the crotonyl-CoA/ethylmalonyl-CoA/hydroxybutyryl-CoA (CETCH) cycle, the reductive glycine pathway (rGlyP), the synthetic acetyl-CoA (SACA) pathway, the PyrS-PyrC-glyoxylate cycle and the C4-PyrC-alanine malonyl-CoA-oxaloacetate-glyoxylate cycle.

The cellular resources required by each of the carbon fixation pathways vary from one to another and determines their relative efficiency. The WLP is considered one of the most efficient carbon fixation pathways since it requires the least amount of ATP and reducing equivalents to fix a given amount of CO₂. The ability to fix CO₂ into acetate makes acetogenic microorganisms an attractive biocatalytic platform for the conversion of CO₂ into useful chemicals, fuels and materials fit for industrial purposes. However, the carbon fixation pathways found in nature cannot be exploited for industrial purposes due to bioenergetic constraints and the relatively low productivity and sub-optimal efficiency of native carbon fixation. Therefore, addressing these limitations and improving the overall efficiency of carbon fixation is of commercial interest.

Carbon fixation has been described in the literature as being organised into a dual-modular architecture, with the key module being the carbon fixation pathway, and the second module being the energy module. Publications in which the inefficiencies of native carbon fixation pathways have been addressed include the following: US 2019/0211342 A1, US 2018/0223317 A1 and U.S. Pat. No. 10,801,045 B2; and review articles by Gong et al. [2] and Zhao et al. [3]. US 2019/0211342 A1 discloses an energy module supplying both reducing power and ATP to support the activity of a non-native CBB pathway in recombinant microorganisms capable of carbon fixation, whereby the source of ATP and reducing power is amassed. US 2018/0223317 A1 and U.S. Pat. No. 10,801,045 B2 disclose a metabolic architecture consisting of an energy module and a carbon fixation module for endowing chemoautotrophic capabilities to autotrophic or mixotrophic microorganisms. The energy module is stated to comprise one or more energy conversion pathways that use energy from inorganic energy sources to specifically produce reduced cofactors, however no consideration is given to the generation of ATP. Gong et al. [2] review the strategies and approaches for improving carbon fixation efficiency by introducing novel energy supply patterns that provide both ATP and reducing equivalents concomitantly. Additionally, Zhao et al. [3] review both synthetic and natural carbon fixation pathways, and discuss different energy supply modules that provide both ATP and reducing equivalents concomitantly for use in engineering carbon fixation pathways.

Therefore, it can be seen that developing new, non-naturally occurring and increasingly productive systems for carbon fixation would provide a contribution to the art. Systems for CO₂ fixation could prove useful in the reduction of environmental CO₂ levels, by generating acetyl-CoA for the renewable generation of industrially-important organic compounds, vitamins, proteins, fats and carbohydrates.

### SUMMARY OF THE INVENTION

The present invention relates to a system for efficient carbon fixation using the components necessary for the generation of acetyl-CoA from an inorganic carbon source such as CO₂. The system allows for increased efficiency of carbon fixation by the use of mechanisms for generating ATP and reducing equivalents that operate independently.

In a first aspect of the invention, there is a system for the generation of acetyl-CoA, comprising the components necessary for the biochemical conversion of an inorganic carbon source into acetyl-CoA, wherein said components comprise:
i. a source of reducing equivalents;
ii. a light-dependent ion pump that generates an electrochemical ion gradient independently of the generation of reducing equivalents; and
iii. a redox-dependent ion pump that generates an electrochemical ion gradient independently of a net change in the number of reducing equivalents.

In a second aspect of the invention, there is a genetically modified acetogen comprising a recombinant rhodopsin, wherein the genetically modified acetogen has an enhanced ability to produce acetyl-CoA.

In a third aspect of the invention, there is a method for generating acetyl-CoA, comprising providing an inorganic carbon source to the system of the first aspect of the invention for the generation of acetyl-CoA, under suitable biochemical conditions.

In a fourth aspect of the invention, there is a method for environmental CO₂ fixation using the system of the first aspect of the invention for the generation of acetyl-CoA.

In a fifth aspect of the invention, there is a method of using the system of the first aspect of the invention for generating acetyl-CoA as a precursor to generate organic compounds, vitamins, proteins, fats, carbohydrates, or a combination thereof.

The system for carbon fixation confers environmental benefit, primarily by converting environmental CO₂ into acetyl-CoA, thus reducing environmental CO₂ levels and providing for the renewable generation of industrially important organic compounds and materials, as well as vitamins, proteins, fats and carbohydrates.

### DESCRIPTION OF FIGURES

The invention is described with reference to the accompanying drawings.

FIG. 1 shows the Wood-Ljungdahl pathway (WLP) for the synthesis of acetyl-CoA from gaseous carbon sources. The WLP is composed of two branches: the carbonyl branch and the methyl branch. In each branch, one molecule of CO₂ is reduced. In the methyl branch, a molecule of CO₂ is first reduced to formate and then bound to tetrahydrofolate (THF) generating formyl-THF, requiring one ATP molecule. Formyl-THF is dehydrated to methenyl-THF and sequentially reduced via methylene-THF to yield methyl-THF. In the carbonyl branch, CO₂ is reduced to enzyme-bound CO in a reaction catalysed by carbon monoxide dehydrogenase/acetyl-CoA synthase complex (CODH/ACS). In the final step of the WLP, the CODH/ACS complex catalyses the synthesis of acetyl-CoA from the methyl group generated from the methyl branch and the enzyme-bound CO generated from the carbonyl branch.

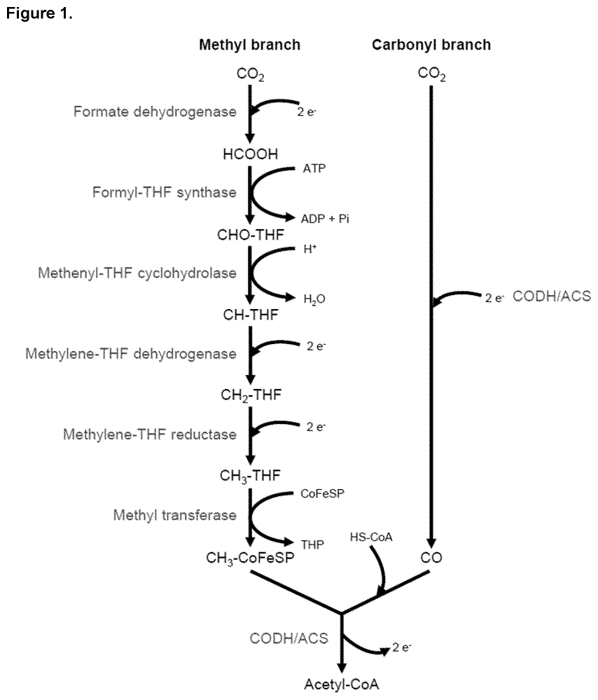

FIG. 2 shows in vivo data demonstrating increased growth of recombinant acetogenic microorganisms when a light-dependent bacteriorhodopsin is present in the system. (A) Comparison of growth on gases in the absence of a light source in wild type Acetobacterium woodii (A. woodii), and A. woodii harbouring one of two empty expression plasmids, one of which was used to express the bacteriorhodopsin tested. (B) All-trans retinal is essential for the functionality of bacteriorhodopsin. A comparison was made of growth on gases in the presence of a light source with wild type A. woodii expressing the bacteriorhodopsin from plasmid pMTL-84151 in the absence of all-trans retinal, wild type A. woodii expressing the bacteriorhodopsin from plasmid pMTL-84151 in the presence of all-trans retinal, and A. woodii harbouring the empty expression plasmid pMTL-84151.

FIG. 3 demonstrates that the functionality of light-dependent bacteriorhodopsins when expressed in wild type A. woodii, and that the activity of bacteriorhodopsin is dependent on the presence of all-trans retinal. Functionality was demonstrated by measuring the change in pH of the medium with cell suspensions of A. woodii expressing bacteriorhodopsin in the presence or the absence of all-trans retinal when illuminated by a light source.

### DETAILED DESCRIPTION

The present invention discloses a system for carbon fixation that decouples the generation of ATP from mechanisms that generate or consume reducing equivalents, both of which are cellular resources required for the conversion of an inorganic carbon source into acetyl-CoA. By modifying existing systems for biological carbon fixation, the resultant system provides for highly efficient carbon fixation compared to dual-modular systems in which ATP and reducing equivalents are generated simultaneously or whereby ATP is generated by a net consumption of reducing equivalents. Accordingly, the metabolism of an inorganic carbon source into chemically useful products may occur extremely efficiently. The system of the present invention comprises three modules rather than two: the carbon fixation pathway, the module for ATP generation, and the module for reducing equivalent generation. The components required for the independent generation of ATP and reducing equivalents may be assembled in both in vitro and cellular systems.

In accordance with the first aspect of the invention, the present invention provides a system for carbon fixation comprising the following components necessary for the conversion of an inorganic carbon source into acetyl-CoA:
i. a source of reducing equivalents;
ii. a light-dependent ion pump that generates an electrochemical ion gradient independently of the generation of reducing equivalents; and
iii. a redox-dependant ion pump that generates an electrochemical ion gradient independently of a net change in the number of reducing equivalents.

As used herein, the term “independently” in the context of the present invention, refers to the separation of mechanisms by which ATP and reducing equivalents are generated simultaneously, or whereby ATP is generated by a net consumption of reducing equivalents. In particular, the generation of an electrochemical ion gradient can occur either concomitantly or independently from the generation of reducing equivalents or consumption of reducing equivalents. An example of the former is the electron transport chain in plants, wherein photosystems use light energy to oxidise water while simultaneously generating an electrochemical ion gradient and the generation of reducing equivalents in the form of NADPH. The mitochondrial electron transport chain is an example of where reducing equivalents are consumed (oxidised) to generate an electrochemical ion gradient. The multi-subunit ferredoxin-NAD⁺ oxidoreductase (Rnf) complex found in acetogens is an example of how an electrochemical ion gradient can be generated independently from the generation of new reducing equivalents, or net consumption of reducing equivalents. The Rnf complex couples the transfer of electrons from Fd²⁻ to NAD⁺ with the transport of ions across the membrane. Although a redox reaction is used to generate an electrochemical ion gradient, there is no net increase or decrease in reducing equivalents. Further examples include cyclic electron transfer in purple non-sulphur bacteria and the action of microbial rhodopsins, both of which use light energy to pump ions across a membrane generating an electrochemical ion gradient independently from the generation or net consumption of reducing equivalents.

As used herein, the term “components” and “biochemical components” are used interchangeably, and in the context of the present invention, refer to any biological macromolecule such as but not limited to proteins or protein complexes that form constituent elements of a biochemical pathway, responsible for the catalysis of enzymatic steps within said biochemical pathway. For example, components necessary for the conversion of an inorganic carbon source into acetyl-CoA may include, but are not limited to, formate dehydrogenase, formyl-tetrahydrofolate (formate-THF) synthase, methenyl-tetrahydrofolate (methenyl-THF) cyclohydrolase, methylene-tetrahydrofolate (methylene-THF) dehydrogenase, methylene-THF reductase, methyl transferase, and carbon monoxide dehydrogenase/acetyl-CoA synthase (CODH/ACS) via the WLP. The term “components” and “biochemical components” may also refer to any biological macromolecule such as but not limited to proteins or protein complexes, non-biological organic compounds or inorganic compounds that form constituent elements of the module for ATP generation or the module for reducing equivalent generation.

In one embodiment, components may be modified such that their enzymatic capacities are enhanced to increase the productivity of a system for carbon fixation. Based on synthetic biology, the carbon fixation pathway, the module for ATP generation, and the module for reducing equivalent generation may be redesigned. Components of each module can be freely combined or modified to develop increasingly productive systems for carbon fixation that offer higher efficiencies and productivities than those found in nature. Typical modifications include targeting low turnover, rate-limiting enzymes. For example, carbon fixation pathways employed by autotrophic organisms are mainly limited by carboxylase enzymes. Therefore, strategies to improve productivity of carbon fixation include replacing rate-limiting enzymes with more efficient homologs, reconstructing hybrid enzymes, or selecting more efficient mutant versions of the rate-limiting enzyme [4-7]. Additional modifications can include overexpression of key enzymes [8]. Further, creating novel systems for carbon fixation by combining the components of other pathways provides several avenues for increasing the productivity of carbon fixation. In a preferred embodiment, flavin based electron bifurcation (FBEB) is used to couple the endergonic reduction of a low potential electron carrier (such as ferredoxin) with a higher potential electron carrier (such as nicotinamide adenine dinucleotide (NADH)) to the exergonic reduction of an intermediate in the carbon fixation pathway with NADH (or similar).

As used herein, the term “system” in the context of the present invention, refers to an assembly of the components necessary for the biochemical conversion of an inorganic carbon source into acetyl-CoA. In one embodiment, the system may be an in vitro system. As used herein, the terms “in vitro”, “acellular” and “cell-free” may be used interchangeably and refer to the assembly of the described components into a system in which the components necessary for the biochemical conversion of an inorganic carbon source into acetyl-CoA are provided to the system. In an in vitro system, the components necessary may be outside their normal biological context. Examples of an in vitro system include, but are not limited to, systems enclosed by a vesicle (including but not limited to exosomes, microvesicles, giant unilamellar vesicles), liposome or microdroplet, which may be composed of carbohydrates, peptides or fatty acids, phospholipids, polymers, or combinations thereof. Further, systems may be enclosed by non-naturally occurring organic compounds or inorganic compounds that may or may not self-assemble and enclose soluble components of the system while also providing a hydrophobic region such that integral or transmembrane proteins or complexes can be inserted into the wall of the membrane.

In another embodiment, the system may be an in vivo system. As used herein, the terms “in vivo” and “cellular” may be used interchangeably and refer to a system in which the components necessary for the biochemical conversion of an inorganic carbon source into acetyl-CoA are already present within the system. Examples of an in vivo system include, but are not limited to, systems enclosed by a semi-permeable barrier, for example, a lipid bi-layer such as a cell membrane. In one embodiment, the system may be enclosed by the membrane of a living cell, such as a bacterial cell.

As used herein, the term “inorganic carbon source” in the context of the present invention, refers to any simple compound wherein the carbon atom is chemically bonded to an element or elements other than hydrogen. For example, an inorganic carbon source may include, but is not limited to, atmospheric carbon generated by natural or industrial manufacturing processes, transportation, or fossil fuel combustion. Examples of inorganic carbon according to the invention may include, but are not limited to, CO₂, CO, carbides, carbonates, and cyanides. However, the preference is for the system of the present invention to convert CO₂ to acetyl-CoA.

As used herein, the term “reducing equivalent” in the context of the present invention, refers to any number of chemical species capable of donating its electrons to an electron acceptor. The terms “reducing equivalent”, “reducing agent”, “electron carrier” and “electron donor” may be used interchangeably and refer to the formal transfer of electrons from one species to another, such that the electron donor becomes oxidised, and the electron acceptor becomes reduced. For example, reducing equivalents that may be generated according to the invention include, but are not limited to, NADH, nicotinamide adenine dinucleotide phosphate (NADPH), flavin adenine dinucleotide (FADH₂), H₂, Fd²⁻, and reduced flavodoxin (Fld^{Hq}) (Table 1). However, the preference is for the system of the present invention to generate NADH, NADPH and Fd²⁻.

As used herein, the term “net change” in the context of the present invention, refers to the pool of reducing equivalents, such that there has been an increase or decrease in the total number of reducing equivalents. In the context of a redox-dependant ion pump, the term “net change” specifically refers to ion pumps that generate an electrochemical ion gradient by mechanisms that are independent of the consumption or generation of reducing equivalents, such that there is no net change in the number of reducing equivalents. For example, the Rnf complex consumes reduced ferredoxin (Fd²⁻) while also generating NADH.

TABLE 1
Cellular cofactors, coenzymes, proteins and organic compounds
acting as redox mediators or involved in redox reactions, flavin
based electron bifurcating (FBEB) redox reactions or quinone
based electron bifurcating (QBEB) redox reactions.

| Name | Type | Symbol (reduced) |
|---|---|---|
| 8-hydroxy-5-deazaflavin | Coenzyme | H₂F₄₂₀ |
| Adrenodoxin | Iron-sulphur protein | Adx²⁻ |
| Cytochrome complex (4 | Heme protein | Cyt c |
| classes) | | |
| Flavin adenine dinucleotide | Coenzyme | FADH₂ |
| Ferredoxin | Iron-sulphur protein | Fd²⁻ |
| Ferrocytochrome c | Iron-heme center | Cyt.c[Fe^{II}] |
| Flavodoxin | Flavoprotein | Fld^{Hq} |
| Flavin mononucleotide (FMN) | Coenzyme | FMNH₂ |
| Formate | Organic compound | CH₂O₂ |
| Hydraquinone | Coenzyme | QH₂ |
| Dihydrogen | Inorganic compound | H₂ |
| Menaquinone (vitamin K) | Vitamin | MKH₂ or MQH₂ |
| Methyl viologen | Organic compound | V^{red} |
| Nicotinamide adenine | Coenzyme | NADH |
| dinucleotide | | |
| Nicotinamide adenine | Coenzyme | NADPH |
| dinucleotide phosphate | | |
| Plastocyanin | Copper protein | PC^{red} |
| Plastoquinones | Quinone | PQH₂ |
| Putidaredoxin | Iron-sulphur protein | Pdr^{red} |
| Quinone | Coenzyme | Q/QH₂ |
| Riboflavin | Vitamin | RBFH₂ |
| Rieske proteins (multiple) | Iron-sulphur protein | Rp²⁻ or ISP²⁻ |
| Rubredoxin | Iron-sulphur protein | Rd²⁻ |
| Semiquinone | Coenzyme | SQ/SQH₂ |
| Thioredoxin (Trx) | Iron-sulphur protein | h²⁻ |
| Ubiquinone (Coenzyme Q) | Coenzyme | UQH₂ (CoQ₁₀) |

In accordance with the invention, the module for generating reducing equivalents consists of biotic and/or abiotic components that facilitate the transfer of electrons from an external source to a reducing equivalent. External electron sources can take the form of organic compounds such as but not limited to formate, inorganic compounds such as but not limited to hydrogen or ammonia, as well as electrical systems providing a current via a cathode, the latter is an example of microbial electrosynthesis (MES). As used herein, the term “biotic” in the context of the present invention, refers to any naturally occurring, recombinant, or synthetic biochemical component. Examples of biotic components that may be used to generate reducing equivalents include, but are not limited to, electron bifurcating enzymes, oxygen-tolerant hydrogenases, formate dehydrogenases, carbon-monoxide dehydrogenases, protein nanowires, and redox mediators.

In a preferred embodiment, the invention incorporates an electron bifurcating enzyme to generate reducing equivalents. As used herein, the term “electron bifurcating enzyme” in the context of the present invention, refers to enzymes that oxidise one electron donor and deliver the electrons simultaneously to two different electron acceptors, whereby reduction of one accepter is exergonic and is tightly coupled to the endergonic reduction of the second acceptor. In a preferred embodiment, the source of reducing equivalents may be an electron bifurcating enzyme selected from Table 2 that the system can make use of for the generation of reducing agents. For example, Fd²⁻ is generated through the activity of an intracellular electron bifurcating hydrogenase, which oxidises hydrogen gas to generate Fd²⁻ and NADH. However, the preferred electron bifurcating enzymes of the inventions are the NADP⁺ and ferredoxin dependent [FeFe]hydrogenase, HytA-E, native to C. autoethanogenum NAD⁺ and ferredoxin dependent [FeFe]hydrogenase, HydABCD, native to A. woodii to generate reducing equivalents.

In another embodiment, the reducing equivalents may be generated by an oxygen-tolerant hydrogenase. As used herein, the term “oxygen-tolerant hydrogenase” in the context of the present invention, refers to oxygen-insensitive enzymes that are capable of catalysing H₂ oxidation to water (H₂O) to generate reducing equivalents, under aerobic conditions while avoiding oxygenation and destruction of the active site. In one mechanism accounting for this property, membrane-bound hydrogenases accommodate a pool of electrons that allows an oxygen molecule to be converted rapidly to H₂O. Examples of oxygen-tolerant hydrogenases include the [NiFe]-hydrogenase from Ralstonia eutropha H16 and the NAD⁺-reducing [NiFe]-hydrogenase from Hydrogenophilus thermoluteolus.

In another embodiment, the reducing equivalents may be generated by a formate dehydrogenase. As used herein, the term “formate dehydrogenase” in the context of the present invention, refers to enzymes capable of catalysing the oxidation of formate to CO₂ with the concomitant reduction of NAD⁺ to NADH or NADP⁺ to NADPH. Examples of formate dehydrogenases include the NADP(H)-dependent [FeFe]-hydrogenase (Hyt) complex from C. autoethanogenum, the NADH-dependent formate dehydrogenase/heterodisulfide reductase (Fdh) complex from Methanococcus maripaludis (HdrABC/FdhAB) and the NADH-dependent formate dehydrogenase (Hyl) complex from Clostridium acidurici.

In another embodiment, the reducing equivalents may be generated by a carbon-monoxide dehydrogenase. As used herein, the term “carbon-monoxide dehydrogenase” in the context of the present invention, refers to enzymes capable of catalysing the reversible oxidation of CO to CO₂. Two classes of carbon-monoxide dehydrogenases exist: Cu,Mo-carbon-monoxide dehydrogenases and Ni,Fe-containing carbon-monoxide dehydrogenases.

In another embodiment, the reducing equivalents may be generated by a protein nanowire. As used herein, the term “protein nanowire” in the context of the present invention, refers to electrically conductive appendages produced by a number of bacteria most notably from, but not exclusive to, the Geobacter and Shewanella genera. Protein nanowires are used for generating reducing equivalents from electrical energy as in MES.

In another embodiment, the reducing equivalents may be generated by a redox mediator. As used herein, the term “redox mediator” in the context of the present invention refers to macromolecules such as proteins and organic compounds involved in the transfer of electrons from external electrochemical sources to reducing equivalents. Redox mediators may be soluble, or membrane bound and are involved in both indirect and direct extracellular electron transfer (EET). Examples of redox mediators includes but is not limited to heme proteins such as cytochromes, flavin based proteins, iron-sulfur proteins, and FMN or quinone based coenzymes (Table 1).

In another embodiment, the reducing equivalents may be generated by abiotic components. As used herein, the term “abiotic” in the context of the present invention, refers to any non-biologically relevant organic compound or inorganic compound. Abiotic compounds may be used for the generation of reducing equivalents from electrical or electromagnetic (light) energy and may be used to facilitate direct or indirect EET. Examples of abiotic components that may be used to generate reducing equivalents include, but are not limited to, allotropic carbon, inorganic compounds, inorganic semiconducting materials, and redox mediators. Abiotic components may be manufactured through a variety of approaches including by not limited to chemical approaches or through deposition such as in 3D printing.

In another embodiment, the reducing equivalents may be generated by an allotropic carbon component. As used herein, the term ‘allotropic carbon’ in the context of the present invention, refers to carbon-based materials. Examples of allotropic carbon include but are not limited to single-wall carbon nanotubes (SWCNTs), multi-wall carbon nanotubes (MWCNTs), graphene, and carbon felt. Allotropic carbon is frequently used for the generation of reducing equivalents by facilitating direct EET in MESs.

In another embodiment, the reducing equivalents may be generated by an inorganic compound. As used herein, the term “inorganic compound” in the context of the present invention, refers to precious and non-precious metals typically used as cathodic materials in MES. Examples of precious metals include but are not limited to palladium and silver. Examples of non-precious metals include but are not limited to cobalt-phosphate, copper and nickel. Inorganic compounds are frequently used for the generation of reducing equivalents in the form of hydrogen, facilitating indirect EET in MES.

In another embodiment, the reducing equivalents may be generated by an inorganic semiconducting material. As used herein, the term “inorganic semiconducting material” in the context of the present invention, refers to a non-carbon based materials such as silicon, gallium or arsenide with an intermediate level of conductivity between that of an insulator and that of most metals. Examples of inorganic semiconducting materials include, but are not limited to, cobalt phosphate (CoP) Inorganic semiconducting materials may also be used as light-harvesting semiconducting materials. The term “light-harvesting semiconducting material” in the context of the present invention, refers to materials with an intermediate level of conductivity between that of an insulator and that of most metals that generate reducing equivalents when illuminated with light. Examples include, but are not limited to, cadmium sulphide semiconducting nanoparticles, silicon nanowires, quantum dots, indium phosphate, and composites of perylene diimide derivative (PDI) and poly(fluorene-co-phenylene) (PFP).

In another embodiment, the reducing equivalents may be generated by a redox mediator. As used herein, the term “redox mediator” in the context of the present invention, refers to organic compounds which can be reduced by external electron sources, or facilitate direct electron transfer to be used to shuffle electrons from an external environment to a cell, enabling electron uptake into cells. Redox mediators can increase the availability of reducing equivalents in the system by enabling the uptake of electrons directly from a cathode via EET as in MES. An example of an abiotic redox mediator includes but is not limited to methyl viologen.

In accordance with the invention, reducing equivalents may be utilised by proteins dependent on them for their activity. For example, NADH-dependent reductase and an NADPH-dependent reductase couple the respective oxidation of NADH and NADPH to the reduction of a substrate. In the methyl branch of the WLP, formyl-THF is dehydrated to methenyl-THF and then sequentially reduced via methylene-THF to yield methyl-THF. The reduction of methenyl-THF to methylene-THF is carried out by both a NADH-dependent methylene-THF dehydrogenase AND an NADPH-dependent methylene-THF dehydrogenase. Further, the reduction of methylene-THF to methyl-THF is preferentially carried out by an electron bifurcating methylene-THF reductase or by an NADH-dependent methylene reductase.

TABLE 2
Enzymes exhibiting flavin-based electron bifurcation.

| | Example Sources | | |
|---|---|---|---|
| | (Genus and/or | | |
| Complex/Enzyme Name | species) | Function | Cofactor Specificity |
| Bcd/EtfAB (Butyryl-CoA | C. kluyveri | Reduction of | Fd + 2 NADH + crotonyl-CoA → Fd²⁻ + |
| dehydrogenase/electron | | crotonyl-CoA | 2 NAD⁺ + butyryl-CoA |
| transfer flavoprotein) | | | |
| CarCDE/EtfAB (Caffeyl-CoA | A. woodii | Caffeate | Fd + 2 NADH + caffeyl-CoA → hydrocaffeyl-CoA + |
| reductase/electron transfer | | reduction | 2 NAD⁺ + Fd²⁻ |
| flavoprotein) | | | |
| Fdh (Formate dehydrogenase/ | M. maripaludis | Formate or dihydrogen | 2 formate (or 2H₂) + CoMS-SCoB + Fd ⇔ 2 CO₂ (or |
| heterodisulfide reductase) | | oxidation and | 4H⁺) + Fd²⁻ + HSCoM + HSCoB |
| | | disulfide reduction | |
| FixABCX or EtfAB | A. vinelandii or | Nitrogen fixation | 2 NADH + Fld^{Sq} + Q ⇔2 NAD⁺ + Fld^{Hq} + QH₂ OR |
| FixCX(nitrogen fixation/ | R. rubrum | | 2 NADH + Fd + Q ⇔2 NAD⁺ + Fd²⁻ + QH₂ |
| electron transfer | | | |
| flavoprotein) | | | |
| Hdr2ABC (F₄₂₀H₂-dependent | M. acetivorans | Disulfide reduction | 2F₄₂₀H₂ + Fd + CoMS-SCoB ⇔ 2F₄₂₀ + Fd²⁻ + |
| heterodisulfide reductase) | | | HSCoM + HSCoB |
| HydABCD (NADH-dependent | A. woodii | Oxidation of | 2 H₂ + NAD⁺ + Fd ⇔ NADH + Fd²⁻ + 4 H⁺ |
| [FeFe] - hydrogenase) | | dihydrogen | |
| HylABC/FdhF2 (dependent | C. acidurici | Formate oxidation | 2 formate + Fd + NAD⁺ ⇔ 2 CO² + Fd²⁻ + NADH |
| formate dehydrogenase) | | | |
| HytA-E1E2/FdhA (NADPH- | C. autoethanogenum | Oxidation of | 2 H₂ + NADP⁺ + Fd ⇔ NADPH + Fd²⁻ + 4 H⁺ Or: |
| dependent [FeFe]- | | dihydrogen or | 2 formate + NADP⁺ + Fd ⇔ NADPH + Fd²⁻ + 2 CO₂ |
| hydrogenase) | | formate | |
| Ldh/EtfAB (Lactate | A. woodii | Pyruvate reduction | 2 NADH + Fd + pyruvate → lactate + 2 NAD⁺ + Fd²⁻ |
| dehydrogenase/electron | | | |
| transfer flavoprotein) | | | |
| Mvh ([NiFe]hydrogenase/ | M. marburgensis | CoM-S-S-CoB- | 2 H₂ + CoMS-SCoB + Fd → Fd²⁻ + HSCoM + |
| heterodisulfide reductase) | | dependent | HSCoB + 2 H⁺ |
| | | reduction of | |
| | | ferredoxin with H₂ | |
| MetVFD/HdrABC | Moorella | Methylene-THF | 2 NADH + methylene-THF + Fd(eq.*) → 2 NAD⁺ + |
| (Methylene-tetrahydrofolate | thermoacetica | reduction to | methyl-THF + Fd²⁻ *Hypothesised to be a Fd equivalent |
| (H4F) reductase/ | | methyl-THF | |
| heterodisulfide reductase) | | | |
| Nfn or Stn (NADH-dependent | C. autoethanogenum, | Transhydrogenase | Fd²⁻ + NADH + 2 NADP⁺ ⇔ 2 NADPH + Fd + NAD⁺ |
| reduced ferredoxin: NADPH | Sporomusa ovata | | |
| oxidoreductase) | | | |

The system of the present invention requires energy encapsulated in the form of ATP for carbon fixation to occur. ATP generation can occur using membrane-bound ATP synthases (ATPases), which are driven by the flow of ions down an electrochemical gradient spanning the membrane. This gradient is maintained by the action of ion pumps, which store energy in the ions' electrochemical potential by transferring them up their electrochemical gradient. The flow of ions forms a circuit which transfers energy from the ion pumps to the ATPases, where the energy is stored by phosphorylation of adenosine diphosphate (ADP) to ATP. The electrochemical gradient is therefore utilised to generate ATP. The electrochemical gradient may be used to generate ATP independently of the generation or consumption of reducing equivalents.

ATP generation module may occur via one or more ion pumps to generate an electrochemical gradient independently of the generation or consumption of reducing equivalents. As used herein, the term “ion pump” in the context of the present invention, refers to at least two proteins or protein complexes that pump ions across a membrane to generate an electrochemical gradient through mechanisms that are independent of the net generation or consumption of reducing equivalents.

Specifically, it refers to proteins or protein complexes that require energy to transport ions against an electrochemical gradient, from areas of low electrochemical potential to areas of high electrochemical potential.

Ion pumps are selective and dependent on ions of a specific species. For example, proton pumps are optimally adapted to drive the passage of hydrogen ions (protons) across a membrane. Other varieties of ion pump include, but are not limited to, sodium ion (Na⁺) pumps, calcium ion (Ca²⁺) pumps, chloride ion (Cl⁻) pumps, potassium ion (K⁺) pumps, sodium/potassium (Na⁺/K⁺) ion pumps, sodium/hydrogen ion (Na⁺/H⁺) pumps, and potassium/hydrogen ion (K⁺/H⁺) pumps. However, it is known in the art that ion pumps are also capable of pumping ions other than their dependent ion, albeit less efficiently.

In one embodiment, the system of the present invention comprises at least two or more ion pumps that are dependent on ions of the same species, and which the electrochemical ion gradient of that same ion is used by the ATP synthase in the system. For example, at least two or more Na⁺ ion pumps, at least two or more K⁺ ion pumps, or at least two or more H⁺ ion pumps.

In another embodiment, the system of the present invention comprises at least two or more ion pumps that are dependent on ions of different species. For example, a Na⁺ ion pump in combination with an H⁺ ion pump. In the context of a system using ion pumps dependent on ions of different species, an antiporter ion pump is present to convert the electrochemical gradient of one ion species into the electrochemical ion gradient of another species. As used herein, the terms “antiporter”, “exchanger” and “counter-transporter” may be used interchangeably, and in the context of the present invention refer to a protein involved in secondary active transport of two or more different ions across a membrane in opposite directions. The term “secondary active transport” refers to one ion species being moved down its concentration gradient, from an area of high electrochemical potential to an area of low electrochemical potential, providing energy for the transport of a second ion species which is moved against its concentration gradient, from an area of low electrochemical potential to an area of high electrochemical potential. Examples of antiporters may include, but are not limited to, Na⁺/K⁺ ion antiporter and Na⁺/H⁺ ion antiporter. Other types of antiporters will be well known in the art.

In a preferred embodiment, the ion pump is selected from the group comprising light-dependent ion pumps and redox-dependent ion pumps.

The term “light-dependent ion pump” refers to any ion pump in which the mechanism of ion transport is controlled by conformational changes in the protein structure caused by absorption of a photon, allowing for precisely regulated flow of ions in response to light stimuli. Examples of light-dependent ion pumps include retinal-pigmented rhodopsins, such as bacteriorhodopsins, proteorhodopsin, deltarhodopsin, xanthorhodopsin, halorhodopsins, channelrhodopsins, archaerhodopsins, and bacterial sensory rhodopsins. However, the preference is for the invention to use a bacteriorhodopsin.

The term “redox-dependent ion pump” refers to any ion pump in which the transport of an ion against its electrochemical gradient is driven by the free energy released from a redox reaction, such that there is not a net change in the number of reducing equivalents. Examples of redox-dependent ion pumps include Rnf complex and Ech complex. However, the preference is for the invention to use an Rnf complex. Light-dependent and redox-dependent ion pumps may be selected from those known in the prior art, listed in Table 3. However, the preference is for the invention to use a bacteriorhodopsin and an Rnf or Ech complex.

TABLE 3
Enzymes and protein complexes involved in generating an electrochemical ion gradient via ion translocating reactions.

| | Example Sources | lon | | |
|---|---|---|---|---|
| Complex Name | (Genus and/or species) | Dependency | Class | Function |
| Ubiquinol:cytochrome | Ectothiorhodospiraceae, | H⁺ | Redox- | QH2 + 2 cytochrome c (Fe^{III}) + 2 H⁺(in) → |
| c - oxidoreductase | Chromatiaceae | | dependent | Q + 2 cytochrome c (Fe^{II}) + 4 H⁺ (out) |
| (cytochrome bc₁ | | | | |
| complex) in conjunction | | | | |
| with the Type II Reaction | | | | |
| Centre (P870 or P960) | | | | |
| with Coenzyme Q, quinones, | | | | |
| cytochrome c₂ | | | | |
| EchA-F complex | Moorella thermoacetica | H⁺ | Redox- | Fd²⁻ + 2H⁺ ⇔ Fd + H₂ |
| (energy converting | | | dependent | |
| [NiFe]-hydrogenase) | | | | |
| EhaA-T/EhbA-Q/ | Methanosphaera | Na⁺/H⁺ | Redox- | Fd²⁻ + 2H⁺ ⇔ Fd + H₂ |
| MbhA-N (energy- | stadtmanae | | dependent | |
| converting | | | | |
| [NiFe]hydrogenase | | | | |
| complex) | | | | |
| Fpo/HdrDE | Methanosarcinales, | H⁺ | Redox- | F₄₂₀H₂ + MP ⇔ F₄₂₀ + MPH₂ OR Fd²⁻ + |
| (membrane-bound | Methanomassiliicoccus | | dependent | CoMS-SCoB ⇔ Fd + HSCoM + HSCoB |
| heterodisulfide | luminyensis | | | |
| reductase) OR Fpo/ | | | | |
| HdrD | | | | |
| MtrA-G | Methanothermobacter | Na⁺ | Redox- | CH₃-H₄MPT + H-SCoM ⇔ CH₃₋ SCoM + H₄MPT |
| | marburgensis | | dependent | |
| NqrA-F (Na⁺- | Vibrio cholerae | Na⁺ | Redox- | NADH + Q + H⁺ ⇔ NAD⁺ + QH₂ |
| translocating NADH- | | | dependent | |
| quinone | | | | |
| oxidoreductase) | | | | |
| NuoA-N (NADH: | Escherichia coli | H⁺ | Redox- | NADH + ubiquinone ⇔ NAD⁺ + H⁺ + ubiquinol |
| ubiquinone | | | dependent | |
| oxidoreductase) | | | | |
| RnfA-G complex | Acetobacteirum woodii | Na⁺/H⁺ | Redox- | Fd²⁻ + NAD⁺ ⇔ Fd + NADH |
| (ferredoxin: NAD⁺ | | | dependent | |
| oxidoreductase) | | | | |
| Rnf + cytochrome c + | Methanosarcina | Na⁺ + H⁺ | Redox- | Fd²⁻ + CoMS-SCoB ⇔ Fd + HSCoM + HSCoB |
| HdrDE (via | acetivorans | | dependent | OR 2Fd²⁻ + 2F₄₂₀ ⇔ 2 Fd + 2HF₄₂₀ |
| MP/MPH₂) OR Rnf | | | | |
| Archaerhodopsin | Archaea | H⁺ | Light- | Extracellular export of ions |
| | | | dependent | |
| Bacteriorhodopsin | Halobacterium halobium, | Na⁺/H⁺ | Light- | Extracellular export of ions |
| | H. salinarum | | dependent | |
| Halorhodopsin | All domains | Na⁺/H⁺ | Light- | Extracellular export of ions |
| | | | dependent | |
| Proteorhodopsin | Vibrio, Flavobacteria | H⁺ | Light- | Extracellular export of ions |
| | | | dependent | |
| Xanthorhodopsin | Salinibacter ruber | H⁺ | Light- | Extracellular export of ions |
| | | | dependent | |

The system of the present invention requires ATP for carbon fixation to occur. Membrane-bound ATP synthases utilise the electrochemical ion gradient generated by the light-dependent and redox-dependent ion pumps to drive the generation of ATP. Electrochemical potential energy is transferred to the ATP synthase as ions flow down their electrochemical gradient, providing energy for the phosphorylation of ADP to ATP.

In accordance with the invention, the ATP generation module makes use of an ATP synthase to drive ATP synthesis at the expense of an electrochemical gradient. As used herein, the term “electrochemical ion gradient” in the context of the present invention, refers to the change in Gibbs energy associated with the transfer of 1 mol of a membrane-permeable ion across that membrane. It comprises both a chemical (or concentrative) part that accounts for the difference in chemical potential between regions of different ion concentrations, and an electrical part that represents the change in electrostatic potential energy due to a difference in electric potential. The terms “chemical gradient” and “concentration gradient” may be used interchangeably.

The direction and rate of passive ion transport across a membrane is determined by the electrochemical gradient of the ion. Accordingly, an electrochemical gradient can be used for the synthesis of ATP by an ATP synthase. The present invention therefore provides a system in which the electrochemical gradient generated by ion pumps (redox-dependent and light-dependent) is utilised to generate ATP.

ATP synthases are multi-subunit protein complexes found in the inner mitochondrial membrane, bacterial plasma membrane and thylakoid membrane. ATP synthases are classified as F-type (phosphorylation factor), V-type (vacuole), A-type (archaea), P-type (proton) or E-type (extracellular) ATPases based on their functional differences. ATP synthases do not necessarily use H⁺ ions as the coupling ion, and can use the electrochemical ion gradient of other ions, including but not limited to, Na⁺ ions.

As used herein, the term “membrane” in the context of the present invention, refers to a semi-permeable barrier. In one embodiment, the term “membrane” may refer to a lipid bi-layer such as a cell membrane. For example, the membrane of a bacterial cell may enclose components of the system. In another embodiment, the term “membrane” may refer to the enclosure of the system of the present invention in a vesicle (including but not limited to exosomes, microvesicles, giant unilamellar vesicles), liposome or microdroplet, which may be composed of carbohydrates, peptides or fatty acids, phospholipids, polymers, or combinations thereof used in an in vitro system. Encapsulation of biological or system components within a non-self-replicating membrane results in an in vitro system. For example, a liposome encapsulating soluble components. Components of the system of the present invention can be extracted from native strains or recombinant microorganisms and subsequently encapsulated within a membrane to constitute an in vitro system, or the components can be generated in situ using in vitro transcription and translation (IVTT) or cell-free protein synthesis (CFPS). Assembly of an in vitro system can be achieved using sonication, centrifugation, microfluidics or other methods known by those skilled in the art. In another embodiment, the term “membrane” may refer to non-naturally occurring organic compounds or inorganic compounds that may or may not self-assemble and enclose soluble components while also providing a hydrophobic region such that integral or transmembrane proteins or complexes can be inserted into the wall of the membrane.

It is envisaged that the system can be contained within a recombinant microorganism. In one embodiment, the recombinant microorganism is capable of self-replication. In another embodiment, the recombinant microorganism is incapable of self-replication.

In yet another embodiment, the recombinant microorganism is attenuated. As used herein, the term “attenuated” in the context of the present invention, refers to the alteration of a microorganism to reduce its pathogenicity, whilst maintaining its viability. Such an attenuated microorganism is preferably a live attenuated microorganism, although non-live attenuated microorganisms are also disclosed.

As used herein, the terms “recombinant microorganism” and “genetically modified” in the context of the present invention, refer to a strain of bacteria that has undergone genetic engineering such that the bacterial DNA has been altered by the introduction of new DNA, excision of native DNA, or other modifications to its sequence or structure. Recombinant DNA methods commonly involve the introduction of new DNA via a vector, for example, a plasmid. Such methods are well known to those skilled in the art. Use of recombinant strains of bacteria may confer advantageous properties to the bacterial strain, such as the ability to express heterologous proteins, or reprogramming of the host's genetic instructions.

As used herein, the term “microorganism” in the context of the present invention, refers to, but is not limited to, acetogenic microorganisms and non-sulphur purple bacteria. In particular, acetogenic microorganisms may include, but are not limited to, Acetobacterium woodii (A. woodii), Clostridium ljungdahlii, C. carboxidivorans, C. autoethanogenum, Eubacterium limosum and Moorella thermoacetica. In particular, non-sulphur purple bacteria may include, but are not limited to, Rhodobacter sphaeroides and Rhodospirillum rubrum.

Acetogenic microorganisms and non-sulphur purple bacteria may be genetically modified such that they express additional proteins and protein complexes for carbon fixation. For example, redox-dependent ion pumps such as Rnf or Ech complexes are already present in acetogenic microorganisms. Therefore, introduction of a light-dependent ion pump, such as a rhodopsin, results in acetogenic microorganisms that can use both redox-dependent and light-dependent mechanisms to generate an electrochemical ion gradient to drive ATP synthesis. Further, photosynthetic machinery is already present in non-sulphur purple bacteria. Therefore, introduction of a redox-dependant ion pump, such as an Rnf or Ech complex, results in non-sulphur purple bacteria that can use both light-dependent and redox-dependent mechanisms to generate an electrochemical ion gradient to drive ATP synthesis. Additionally, microorganisms may be genetically modified such that photosynthetic machinery is present with a light-dependant ion pump. Finally, microorganisms may be genetically modified such that a combination of photosynthetic machinery, light-dependent ion pumps and redox-based ion pumps is present to generate an electrochemical ion gradient to drive ATP synthesis.

As used herein, the term “photosynthetic machinery” in the context of the present invention, refers to the biological or non-biological components necessary for the capture and storage of light energy in the form of electrochemical potential energy and/or chemical potential energy. The former is achieved through the translocation (pumping) of ions across a membrane against their electrochemical gradient, and the latter is achieved through the reduction of a substrate, reducing equivalent or redox mediator. Components comprising the photosynthetic machinery may include, but are not limited to, the photosynthetic machinery (cytochrome bc1 complex, cytochrome c, quinone, Type II reaction centre, peripheral antennae and/or chlorosomes) of purple sulphur bacteria, ion pumps that require the action of all-trans-retinal (microbial rhodopsins), and organic or inorganic semiconducting materials.

The present invention utilises components of a linear carbon fixation pathway. The preferred system is the WLP, or a modified version thereof. In one embodiment, the WLP is modified such that a recombinant rhodopsin is introduced into an acetogenic microorganism, resulting in a recombinant acetogenic microorganism capable of using light to produce ATP. Preferably, the acetogenic microorganism is A. woodii.

In accordance with the second aspect of the invention, there is a genetically modified acetogen comprising a recombinant rhodopsin, wherein the genetically modified acetogen has an enhanced ability to produce acetyl-CoA. The genetically modified acetogen comprising a recombinant rhodopsin may comprise any of the components of the preceding aspects of the invention necessary to convert an inorganic carbon source into acetyl-CoA. Genetic modification of an acetogen to comprise a rhodopsin (a light-dependent ion pump) therefore results in the generation of an acetogenic microorganism capable of using both redox-dependent and light-dependent mechanisms to generate an electrochemical ion gradient to drive ATP synthesis.

In accordance with the third aspect of the invention, there is a method for generating acetyl-CoA, comprising providing an inorganic carbon source to the system of the first aspect of the invention for the generation of acetyl-CoA under suitable biochemical conditions.

As used herein, the term “biochemical product” in the context of the present invention, refers to any desired end product generated from the acetyl-CoA with commercially or industrially relevant applications. Preferably, desirable biochemical end products may be selected from the compound classes comprising alcohols, aldehydes, alkaloids, alkanes, alkenes, alkynes, natural or synthetic amino acids, amines, aromatics, carboxylic acids, dicarboxylic acids, dienes, diols, esters, ethers, polymeric (ex. polyhydroxyalkanoates) and monomeric (ex. ethylene glycol) chemicals, isoprenoids, polyketides, surfactants, terpenes, terpenoids, sugars, proteins, fats and other secondary metabolites and can be directed into other processes for the purpose of generating, for example, renewable materials or products. In one embodiment, industrial CO₂ emissions from a manufacturing plant may be fed directly into the system such that the desired end product can be fed directly back into the manufacturing plant, thus providing a renewable source of precursor or intermediate for manufacturing processes. In another embodiment, renewable energy can be stored in chemical bonds through the conversion of CO₂ into highly reduced and longer chain organic compounds.

In accordance with the fourth aspect of the invention, there is a method for environmental CO₂ fixation using the system of the first aspect of the invention for the generation of acetyl-CoA. As used herein, the term “environmental CO₂ fixation” in the context of the present invention, refers to the take up of CO₂ from either the envelope of gases surrounding the earth by the system, thus providing a method to reduce atmospheric CO₂ levels, or to the take up of CO₂ from large bodies of saline water, thus providing a method to reduce oceanic CO₂ levels.

In accordance with the fifth aspect of the invention, there is a method of using the system of the first aspect of the invention for generating acetyl-CoA as a precursor to generate vitamins, proteins, fats and carbohydrates. As used herein, the term “precursor” in the context of the present invention, refers to a compound that participates in a chemical reaction to product another compound. In particular, the term “precursor” may refer to acetyl-CoA as an intermediate compound preceding another in a metabolic pathway to generate vitamins, proteins sugars, fats and/or carbohydrates. For example, the CoA moiety of acetyl-CoA comprises a pantothenic acid tail, also known as vitamin B5. Other vitamins include, but are not limited to, vitamin B12, which requires aminolevulinic acid and which acetyl-CoA is biosynthetic precursor for, as well as vitamin B₉ (folate), which is generated via the shikimate pathway from the substrates phosphoenolpyruvate and erythrose-4-phosphate via the shikimate pathway, both of which can be synthesized from acetyl-CoA. Additionally, acetyl-CoA is a precursor for amino acid biosynthesis and by extension, for the production of proteins as it provides direct entry into the citric acid cycle. Acetyl-CoA can be carboxylated to pyruvate and can therefore enter the reverse glycolytic pathway and pentose phosphate pathway. Additionally, acetyl-CoA is the key metabolic precursor for fatty acid biosynthesis. Malonyl-ACP and acetyl-ACP are required for the first step in chain elongation, and both are derived directly from acetyl-CoA. Additionally, the conversion of acetyl-CoA into pyruvate provides entry into gluconeogenesis, enabling carbohydrate anabolism. Accordingly, acetyl-CoA as a precursor could be used to generate vitamins, sugars, fats and/or carbohydrates as a food source in environments not conducive to food generation.

The use of the alternative (e.g., “or”) should be understood to mean either one, both, or any combination thereof of the alternatives. As used herein, the indefinite articles “a” or “an” should be understood to refer to “one or more” of any recited or enumerated component.

### Examples

### Example 1—Evaluating the Effect of Introducing a Bacteriorhodopsin into A. woodii Growing on Gas

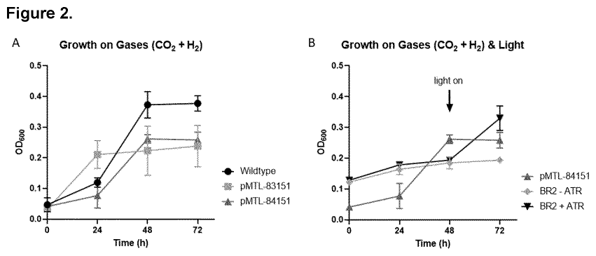

The acetogen A. woodii contains a Na⁺ ion dependent ferredoxin:NAD⁺ oxidoreductase (Rnf) complex which oxidises ferredoxin and reduces NAD⁺, with the concomitant transfer of Na⁺ ions across the membrane, creating an electrochemical gradient that is used to drive a Na⁺ ion specific ATP synthase, for the generation of ATP. To overcome the bioenergetic limitations of the endogenous metabolism (carbon fixation system), it is necessary to bioengineer the host to generate additional ATP for increased growth and biomass formation, or to increase formation of commercial products of interest beyond acetate. One way to achieve this is to increase the generation of the electrochemical gradient. Typically, this is generated by the above mentioned Rnf complex. However, it is also possible to introduce a light-based ion pump such as a bacteriorhodopsin. A Na⁺ ion specific bacteriorhodopsin (BR2) from Krokinobacter eikastus (K. eikastus) was recently reported, which if introduced into A. woodii should increase the generation of the electrochemical gradient, and ultimately ATP formation. Increased ATP formation should result in improved growth/biomass formation, and thus can be used as a proxy to evaluate if the strategy is working (FIG. 2).

(A) Wild type (DSM 1030) Acetobacterium woodii (A. woodii) and the empty expression plasmids pMTL-84151 and pMLT-83151 were used as controls. (B) The bacteriorhodopsin-2 (br2) gene from K. eikastus was cloned into pMTL-84151, and the resultant recombinant plasmid was transformed into A. woodii.

Bacteriorhodopsins requires all-trans retinal (ATR) to be functionally active. Proton-pumping rhodopsins are associated with a retinal pigment that is isomerised from the all-trans state to the 13-cis state after absorption of a photon. Therefore, exogenous addition of ATR to culture media is necessary, as ATR is not naturally synthesised by A. woodii. Strains of A. woodii expressing bacteriorhodopsin in the presence (+ATR) were compared to the wild type and to A. woodii expressing bacteriorhodopsin in the absence (−ATR) of all-trans retinal, which served as a negative control. Pre-cultures of each strain were grown on a defined medium with fructose as the substrate, and inoculated into defined media lacking a carbon source. A gaseous mixture of 50% CO₂ and 50% H₂ was supplied to strains. The light source was applied after 48 hours when maximal growth of the strains was reached. A final optical density at 600 nm (OD₆₀₀) reading was taken after 72 hours.

### Example 2—Measuring In Vivo Activity of Bacteriorhodopsin (BR2)

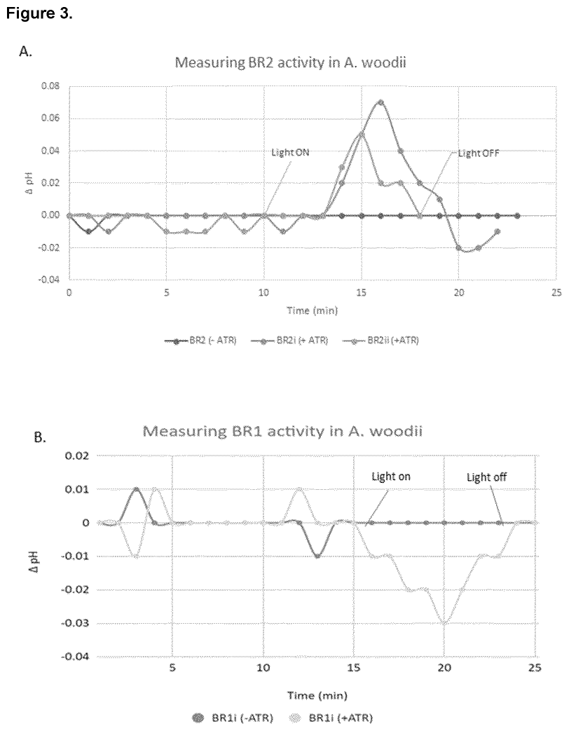

When introducing a heterologous protein into a new host, it is unclear whether the protein is expressed and if the protein is functional. The activity of introduced light-based ion pumps such as bacteriorhodopsins can be measured in vivo by measuring the pH change (ΔpH) of a solution containing cells expressing the protein and illuminated with light. This is one of the methods that is typically used when evaluating the activity of various rhodopsins when expressed in heterologous hosts (FIG. 3).

(3A) The br2 gene was cloned into a pMTL-84151 expression plasmid and transformed into A. woodii. ATR was exogenously added to media to enable functionality of the bacteriorhodopsin. Three clones of A. woodii expressing the BR2 protein were cultured, two of which (BR2i and BR2ii) were grown on media with exogenous ATR supplementation (+ATR). The third clone was cultured in the absence of exogenous ATR supplementation (−ATR), providing a negative control. Cultures were harvested in the mid-exponential growth phase (OD₆₀₀˜0.3) by centrifugation and washed in a buffer (100 mM NaCl, pH 7.2-7.3), then re-suspended in the wash buffer to an OD₆₀₀ of 2.0. The pH of the solution was measured continuously by submerging a pH probe in the solution. The solution was allowed to equilibrate for at least 10 minutes before the light source was applied. The light source emitted light in the range 500-550 nm, and had 5 power settings (0-5). The light power setting applied to BR2i and BR2ii clones was 5 and 4, respectively. (3B) The same protocol was used to evaluate the activity of the br1 gene when introduced into A. woodi using the expression plasmid pMTL-84151. The protein BR1 is proton (H⁺) specific and is have the opposite effect on the pH as BR2, which was validated.

### REFERENCES

1. World Greenhouse Gas Emissions: 2018. (2021). Retrieved 21 Dec. 2021, from https://www.wri.org/data/world-greenhouse-gas-emissions-2018
2. F. Gong, H. Zhu, Y Zhang, and Y. Li (2018) Biological carbon fixation: From natural to synthetic J. CO2. Util., 28; 221-227.
3. T. Zhao, Y Li, and Y. Zhang (2021) Biological carbon fixation: a thermodynamic perspective. Green. Chem. 23:7852-7864.
4. M. T. Lin, A. Occhialini, P. J. Andralojc, et al (2014) A faster Rubisco with potential to increase photosynthesis in crops. Nature. 513; 547-550.
5. T. Genkov, M. Meyer, H. Griffiths, et al (2010) Functional hybrid rubisco enzymes with plant small subunits and algal large subunits: engineered rbcS cDNA for expression in chlamydomonas. J. Biol. Chem. 285; 19833-19841.
6. C. Ishikawa, T. Hatanaka, S. Misoo, et al (2011) Functional incorporation of sorghum small subunit increases the catalytic turnover rate of Rubisco in transgenic rice. Plant. Physiol. 156; 1603-1611.
7. Z. Cai, G. Liu, J. Zhang, et al (2014) Development of an activity-directed selection system enabled significant improvement of the carboxylation efficiency of Rubisco, Protein. Cell. 2014; 1-11.
8. D. M. Rosenthal, A. M. Locke, M. Khozaei, et al (2011) Over-expressing the C3 photosynthesis cycle enzyme Sedoheptulose-1-7 Bisphosphatase improves photosynthetic carbon gain and yield under fully open air CO2 fumigation (FACE). BMC. Plant. Biol. 11 (123).

1. A system for the generation of acetyl-Coenzyme A (acetyl-CoA), comprising components necessary for the biochemical conversion of an inorganic carbon source into acetyl-CoA, wherein said components comprise:
i. a source of reducing equivalents;
ii. a light-dependent ion pump that generates an electrochemical ion gradient independently of the generation of reducing equivalents; and
iii. a redox-dependent ion pump that generates an electrochemical ion gradient independently of a net change in the number of reducing equivalents; and
wherein the electrochemical gradient generated by the ion pumps of (ii) and/or (iii) is utilized to generate ATP.

2. (canceled)

3. The system according to claim 1, wherein the inorganic carbon source is CO2.

4. The system according to claim 1, wherein the components comprise those of a linear carbon fixation pathway.

5. The system according to claim 4, wherein the linear carbon fixation pathway comprises components of the Wood-Ljungdahl pathway (WLP) required to generate acetyl-CoA.

6. The system according to claim 1, wherein said system comprises NADH- and NADPH-dependent reductases and an electron bifurcating reductase.

7. The system according to claim 6, wherein the NADH- and NADPH-dependent reductases are present together with a transhydrogenase that interchanges reducing equivalents from one species to another.

8. The system according to claim 1, wherein the reducing equivalents of (i) are generated by biotic and/or abiotic components.

9. The system according to claim 8, wherein the biotic and/or abiotic components comprise one or more of: electron bifurcating hydrogenase, oxygen tolerant hydrogenase, formate dehydrogenase, carbon monoxide dehydrogenase, inorganic semiconducting material, redox mediator, protein nanowire.

10. The system according to claim 1, wherein the ion pumps of (ii) and (iii) are present as part of a membrane and require energy to pump ions across said membrane from areas of low electrochemical potential to areas of high electrochemical potential, thus generating an electrochemical ion gradient.

11. The system according to claim 10, wherein an ATP synthase uses said electrochemical ion gradient generated by the ion pumps of (ii) and/or (iii) to generate ATP.

12. The system according to claim 11, wherein the ion pumps of (ii) and (iii) and the ATP synthase depend preferentially on ions of the same species.

13. The system according to claim 11, wherein the ion pumps of (ii) and (iii) and the ATP synthase depend preferentially on ions of different species, and an antiporter ion pump is present to convert a chemical ion gradient of one species into a chemical ion gradient of a second species, to increase the electrochemical ion gradient that the ATP synthase is dependent upon.

14. The system according to claim 1, further comprising photosynthetic machinery of purple non-sulphur bacteria.

15. The system according to claim 1, wherein the light-dependent ion pump is a rhodopsin.

16. The system according to claim 15, wherein the rhodopsin is a bacteriorhodopsin, proteorhodopsin, deltarhodopsin, xanthorhodopsin, halorhodopsin, channelrhodopsin, archaerhodopsin, or bacterial sensory rhodopsin.

17. The system according to claim 1, wherein the redox-dependent ion pump is an Rnf or Ech protein complex.

18. The system according to claim 1, wherein said system comprises a recombinant microorganism.

19. The system according to claim 18, wherein the recombinant microorganism is an acetogen.

20. The system according to claim 18 wherein the recombinant microorganism is a purple non-sulphur bacterium.

21. The system according to claim 1, wherein said system is an in vitro system.

22. The system according to claim 1, further comprising biochemical components necessary for converting the generated acetyl-CoA into a biochemical product.

23. The system according to claim 22, wherein the biochemical product is selected from one of the following compound classes: alcohols, sugars aldehydes, alkaloids, alkanes, alkenes, alkynes, natural or synthetic amino acids, amines, aromatics, carboxylic acids, dicarboxylic acids, dienes, diols, esters, ethers, polymeric and monomeric chemicals, isoprenoids, polyketides, surfactants, terpenes, terpenoids, proteins, fats, and other secondary metabolites and/or a combination thereof.

24. A genetically modified acetogen comprising a recombinant rhodopsin, wherein the genetically modified acetogen has an enhanced ability to produce acetyl-CoA, and wherein the genetically modified acetogen comprises the components defined in claim 1.

25. (canceled)

26. The genetically modified acetogen according to claim 24, wherein the recombinant rhodopsin generates an electrochemical ion gradient independently of the generation of reducing equivalents.

27. A method for generating acetyl-CoA, comprising providing an inorganic carbon source to a system according to claim 1, under suitable biochemical conditions.

28. The method according to claim 27, wherein said method further converts the generated acetyl-CoA into a biochemical product.

29. The method according to claim 28, wherein the biochemical product is selected from one of the following compound classes: alcohols, sugars aldehydes, alkaloids, alkanes, alkenes, alkynes, natural or synthetic amino acids, amines, aromatics, carboxylic acids, dicarboxylic acids, dienes, diols, esters, ethers, polymeric and monomeric chemicals, isoprenoids, polyketides, surfactants, terpenes, terpenoids, proteins, fats, and other secondary metabolites and/or a combination thereof.

30. A method for environmental CO₂ fixation using the system of claim 1.

31. A method for using carbon fixation to generate vitamins, proteins, fats, carbohydrates, and/or a combination thereof, wherein said method comprises using a system according to claim 1, wherein the acetyl-CoA is a precursor for the generation of said vitamins, proteins, fats, and carbohydrates.
